(19) United States
(12) Patent Application Publication
(10) Pub. No.: US 2025/0050070 A1
(43) Pub. Date: Feb. 13, 2025

(54) System And Method For Securing Lines

(71) Applicant: Mayo Foundation for Medical Education and Research, Rochester, MN (US)

(72) Inventor: Jillian Amoroso, Cave Cree, AZ (US)

(73) Assignee: Mayo Foundation for Medical Education and Research, Rochester, MN (US)

(21) Appl. No.: 18/720,351

(22) PCT Filed: Dec. 16, 2022
(86) PCT No.: PCT/US2022/081730

§ 371 Date: Jun. 14, 2024

Related U.S. Application Data
(60) Provisional application No. 63/290,195, filed on Dec. 16, 2021.

(51) Int. Cl. A61M 25/02

(52) U.S. Cl. CPC A61M 25/02; A61M 2025/026; A61M 2025/028; A61M 2205/0272

(57) ABSTRACT

A wrap for collecting a plurality of lines, cords, tubes, or wires has been provided. In some implementations, the wrap may include a first surface and a second surface on opposite sides of the first layer, such that one may be viewed as being on the top of the first layer and the other on the bottom of the first layer when the wrap is in an unwrapped state. The first connection mechanism is removably attachable to the second connection mechanism to transition the wrap between the unwrapped state and a wrapped state. The first layer is configured to wrap around a plurality of lines, cords, tubes, or wires when the wrap is in a wrapped state, and a portion of the first layer overlaps another portion of the first layer when the wrap is in a wrapped state.

### CROSS-REFERENCE TO RELATED APPLICATIONS

This application is a national stage filing of PCT/US22/81730 filed on Dec. 16, 2022, which claims priority to U.S. Provisional Application No. 63/290,195 filed on Dec. 16, 2021. Both applications are incorporated by reference for all purposes.

### TECHNICAL FIELD

Aspects of the present disclosure relate to the organization of lines that contain fluids and, in particular, to a system and method for securing lines, tubes, cords and wires.

### BACKGROUND

Lines that are used to transport fluids (e.g., liquids or gases) must be critically organized-especially in medical facilities. Unfortunately, conventional approaches have a variety of shortcomings, including the wasteful time used to organize such lines and the unfortunate pinching that such products may cause.

### SUMMARY

A wrap for collecting a plurality of lines, tubes, cords, and wires has been provided. In some implementations, the wrap may include a first surface and a second surface on opposite sides of the first layer, such that one may be viewed as being on the top of the first layer and the other on the bottom of the first layer when the wrap is in an unwrapped state. The first connection mechanism is removably attachable to the second connection mechanism to transition the wrap between the unwrapped state and a wrapped state. The first layer is configured to wrap around a plurality of tubes when the wrap is in a wrapped state, and a portion of the first layer overlaps another portion of the first layer when the wrap is in a wrapped state.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The phrase “at least one of,” when used with a list of items, means that different combinations of one or more of the listed items may be used, and only one item in the list may be needed. For example, “at least one of: A, B, and C” includes any of the following combinations: A; B; C; A and B; A and C; B and C; and A and B and C. Definitions for certain words and phrases are provided throughout this patent document, those of ordinary skill in the art should understand that in many if not most instances, such definitions apply to prior, as well as future uses of such defined words and phrases.

### BRIEF DESCRIPTION OF DRAWINGS

The various features and advantages of the technology of the present disclosure will be apparent from the following description of particular embodiments of those technologies, as illustrated in the accompanying drawings. It should be noted that the drawings are not drawn to scale; instead, the emphasis is placed on illustrating the principles of the technological concepts. Also, in the drawings the like reference characters refer to the same parts throughout the different views. The drawings depict only typical embodiments of the present disclosure and, therefore, are not to be considered limiting in scope.

FIGS. 1A, 1B, and 1C show conventional products.

FIG. 2 show a typical patent patient transport.

FIG. 3 shows a sleeve 300, according to an embodiment of the disclosure.

FIG. 4 shows a sleeve wrapping around itself, according to an embodiment of the disclosure.

FIGS. 5A and 5B show a clamp, according to an embodiment of the disclosure.

FIGS. 6A and 6B show a clamped sleeve design, according to an embodiment of the disclosure.

FIGS. 7A through 7D show a configuration of using individual sleeves for lines to attract multiple lines together.

FIGS. 8A through 8D show rigid structure sleeves that may be used in particular configurations.

FIGS. 9A and 9B illustrate the placement of a sleeve around a plurality of lines.

FIGS. 10A, 10B, and 10C illustrate a modular nature of layers, according to an embodiment of the disclosure

### DETAILED DESCRIPTION

The figures described below, and the various embodiments used to describe the principles of the present disclosure in this patent document are by way of illustration only and should not be construed in any way to limit the scope of the disclosure. Those skilled in the art will understand that the principles of the present disclosure invention may be implemented in any type of suitably arranged device or system. Additionally, the drawings are not necessarily drawn to scale. Select figures are distorted specifically for purposes of illustration.

Critically ill patients require transportation to diagnostic exams and invasive procedures performed outside the Intensive Care Unit (ICU) or hospital room. In transport, the cords and lines often tangle and become disorganized. This has been called “spaghetti syndrome”. The lack of cord and line management can lead to an unsafe and disorganized environment. Critical monitoring may be interrupted, putting the patient in a temporary unmonitored state; the integrity of IV infusions can be compromised when IV lines are disconnected to detangle; and nurse experiences may be suboptimal due to the disorganized, inefficient environment.

A retrospective observational study performed at the National Taiwan University Hospital (NTUH) analyzed intra-hospital transportation (IHT) associated patient safety events. IHT incidences recorded included process events (e.g., delayed departure and wrong patient transported), physiologic changes including medication reaction and hypotension and equipment events such as, an insufficient supply of oxygen and tube and line removal. Although not discussed in detail, the incidence of tube and line removal was 5.8%. The spaghetti syndrome can make nursing care very difficult, leading to potentially fatal outcomes due to errors. In response, the authors were responsible for color-coding venous, arterial, and pulmonary artery lines. Previous attempts at solving this problem include the creation of a small device that separated each line in independent slots. More currently, a flexible, sleeved-strap banding device that is fixed to a stationary surface was created to manage spaghetti syndrome in critical care.

Aside from line entanglement during transportation, strangulation by medical lines in the pediatric population has been reported. One report was of the devastating case of an 11-month-old boy who was found with IV tubing wrapped around his neck while admitted to the hospital, which unfortunately took his life. A cross-sectional descriptive study was conducted to describe the prevalence and severity of medical line entanglements, including 486 pediatric patients having multiple medical lines including infusion lines, tubes and wires were observed and scored using a severity score. Results revealed that at least one-third of the pediatric patients had at least one entanglement and 11% of children had medical lines tangled with each other.

Critical time is spent detangling lines and cords which may lead to disruptions and delays in patient flow processes in diagnostic and interventional areas. Accidental disconnection or dislodgement of IV lines can cause a multitude of complications including interruption to treatment, air embolism, infection, bleeding, discomfort to the patient, and additional nursing care.

Given the above concerns, embodiments disclose a novel stabilization device to secure and manage IV lines/monitor cords during patient transportation.

While certain embodiments will be described with reference to medical fields, other fields can also avail from teaching of the disclosure. This disclosure may benefit any scenario where fluid lines need to be secured and/or organized. Non-limiting examples include aviation, oil and gas, computer server farms (especially ones dealing with fluid cooling systems). Also, while “fluid” lines may particularly benefit from select embodiments, any other type of line that might need to be secured and/or organized can benefit from select disclosures. Non-limiting examples include electrical power lines. As used herein, a “line” may refer to cords, wires, and tubes.

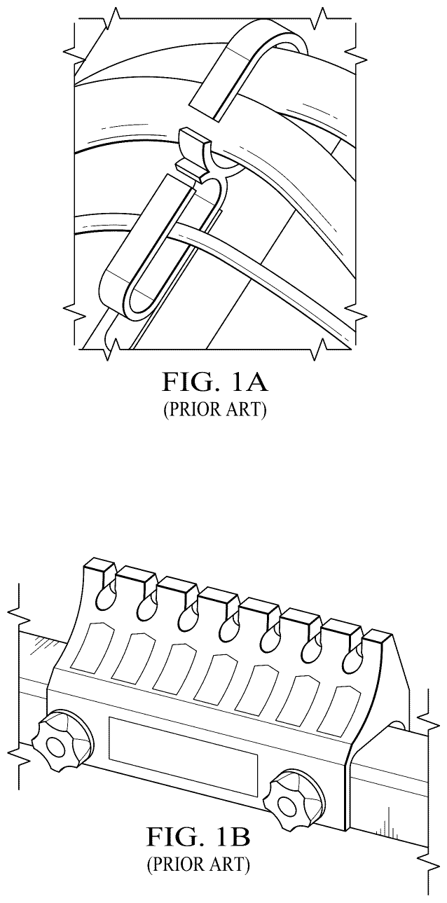

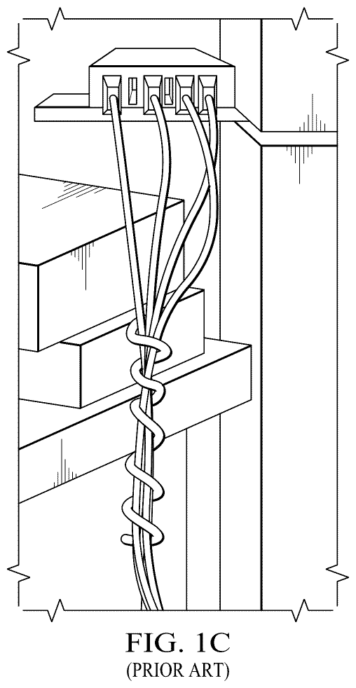

FIGS. 1A, 1B, and 1C show conventional products for medical fluid lines. FIG. 1A shows a product marketed as iLine select. The iLine Select product is a plastic clip that has an adhesive back to allow mounting a patient's bed or other surface. FIG. 1B shows a product marketed as IV Guard. The IV Guard product is molded array of plastic clips that secure to a patient bed (or other item) using screw clamps.

FIG. 1C shows gear ties. The gear ties are an otherwise straight bendable line that holds cords in place through several revolutions.

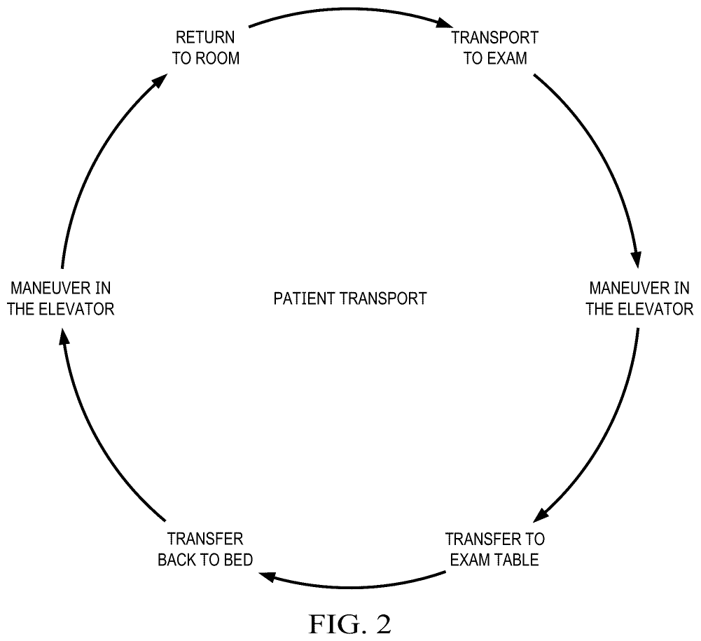

The conventional products of FIGS. 1A, 1B, and 1C suffer from a variety of shortcomings. One of the principal shortcomings amongst these varieties is in their inability to be used in the multi-faceted patient transport setting, which is shown with reference to FIG. 2.

FIG. 2 shows a typical patent patient transport where, for example, a patient will be transported from their room en route to an exam room where they may need to be maneuvered through elevators. Once in the exam room, they may need to be transferred to a table and then back to a bed. Then, in route back to their room, they must (again) maneuver through the elevator before arriving in their room again. In these settings, when the use of traditional products is utilized, a cumbersome and time-consuming engaging and disengaging occurs. For example, the products of FIGS. 1A and 1B are physically hooked to the bed and not portable. To leave the room, the IVs must be disengaged from such products. While the gear ties of FIG. 1C are portable, they are not easy to engage and disengage. They take time. When the process of FIG. 2 is repeated over and over, the otherwise minimal time involved with such engaging and engaging with the products of FIGS. 1A, 1B, and 1C adds up.

Accordingly, embodiments of the disclosure are directed to alleviating concerns with the use of the prior products. In the description of the various embodiments, while IV lines may avail from the disclosures, a variety of lines-including those used in medical and non-medical settings-can avail also avail from this disclosure. Again, non-limiting examples include aviation, oil and gas settings, computer, and electrical fields.

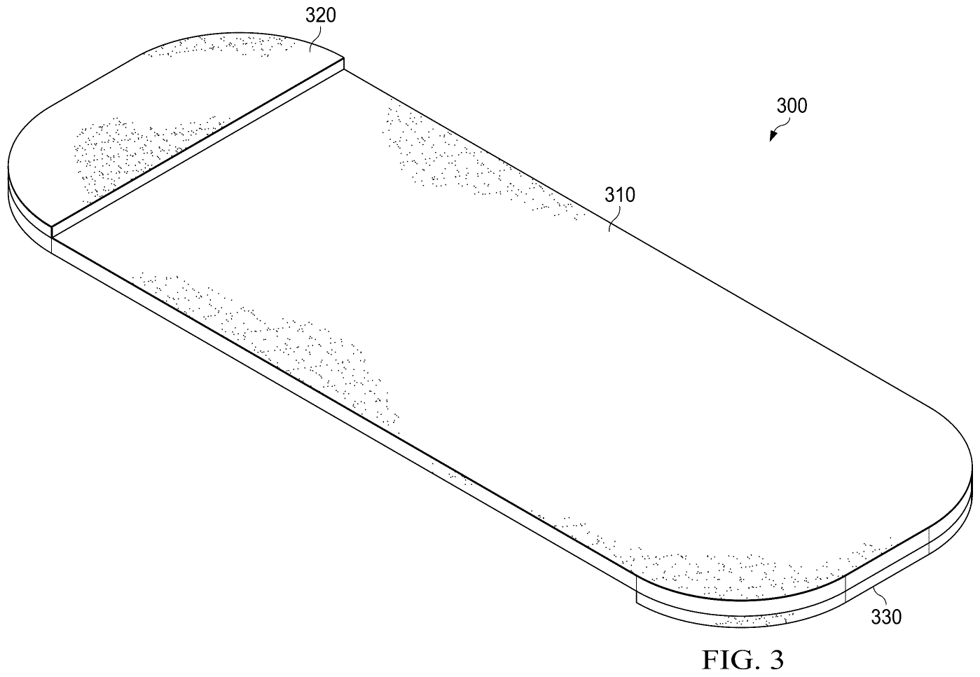

FIG. 3 shows a sleeve 300, according to an embodiment of the disclosure. The sleeve has a wrapping portion 310 and connecting portions 320 and 330. In particular configurations, the wrapping portion 310 may be made of a flexible material. A non-limiting example is neoprene; however, other materials may be utilized including, but not limiting to lycra, spandex, and fabrics. The wrapping portion 310 holds the cords, lines, wires, and tubes in place, while avoiding the pinching of a flow of fluid through the lines.

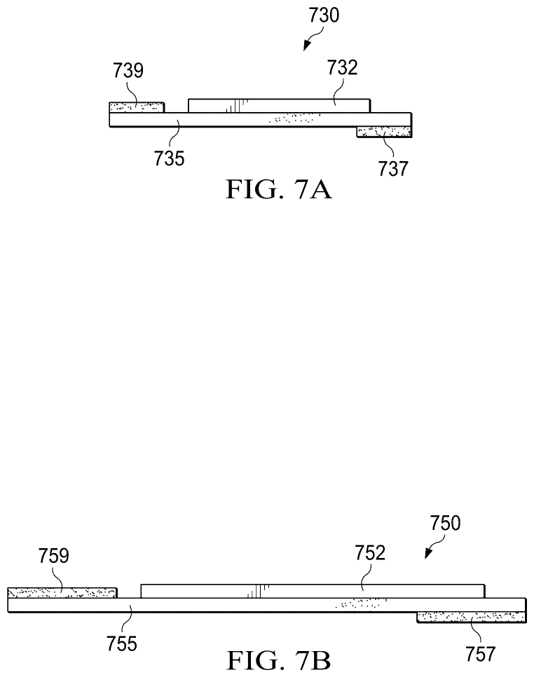

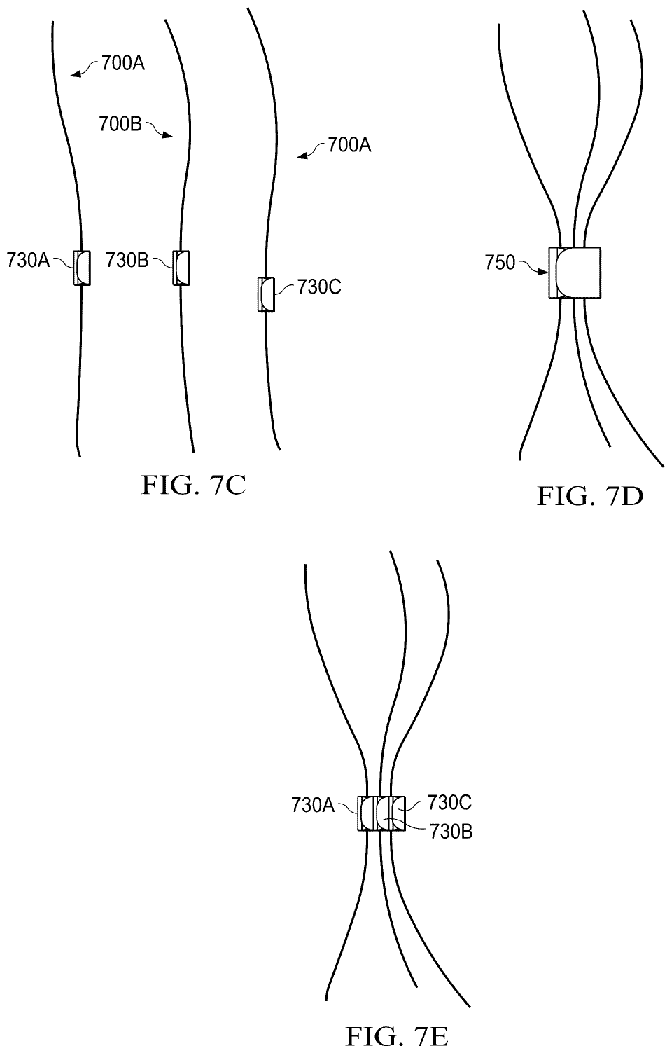

While not seen from the view of FIG. 3, in certain configurations the wrapping portion 310 may be equipped with configurations and/or materials to be used to urge the lines being secured into position. As a non-limiting example, magnets may be placed on the wrapping portions 310 that attract a corresponding magnet that may be placed on the lines being secured. The lines themselves may have temporary cuffs on them that help the line to “snap” into place on the wrapping portion. Further details of such a configuration are shown below with reference to FIGS. 7A through 7D.

The connecting portions 320, 330 assist the wrapping of the sleeve of the lines being secured. In some configurations, hook and loop fasteners may be used. Other materials for connecting portions include, but are not limited to magnets and a flexible loop that attaches, for example, around a knob or hook.

In particular configurations, the wrapping portion 310 may be configured to wrap around the lines in either direction. That is, the surface of wrapping portion 310 seen in FIG. 3 may come in contact with the lines in wrapping. Or, the opposite surface of the wrapping portion (which is not seen in FIG. 3) may come in contact with the lines in wrapping. In some configurations, each of the respective surfaces of the wrapping portion 310 may have different configurations to allow different uses of the wrap-depending, for example, on the direction of the wrapping.

The large shape of the connecting portion 330 allows for differing size of a rolling around the cords, lines, wires, and tubes-depending on where the respective connection portion 320 connect the connecting portions.

In some configurations, the material used for the wrapping portion 310 may be reinforced to resist a general movement. That is, once put in place a general movement is resisted. A non-limiting example is wire embedded in the material. The wire can generally hold shape while the material can flexibly interact (and not pinch the lines). Using the reinforcement in some configurations may reduce (or eliminate) the use of the connecting portions.

FIG. 4 shows a sleeve wrapping around itself, according to an embodiment of the disclosure. Again, as referenced above, the sleeve in particular configurations may alternatively wrap in a different direction, exposing an opposite surface of the sleeve to the lines—instead of the one shown in FIG. 4.

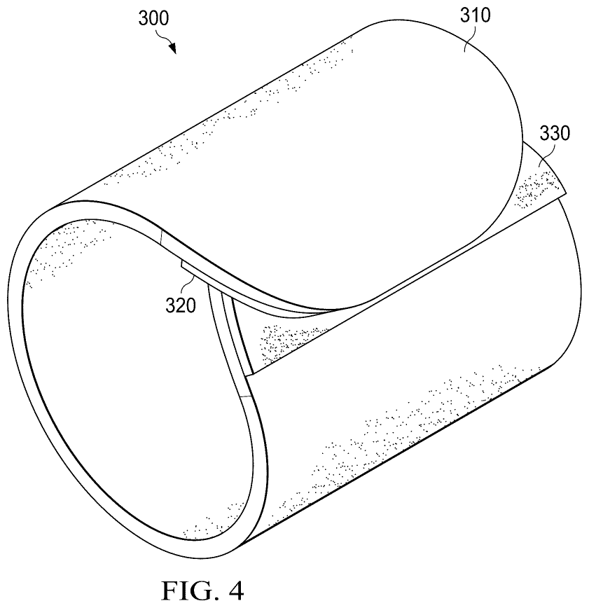

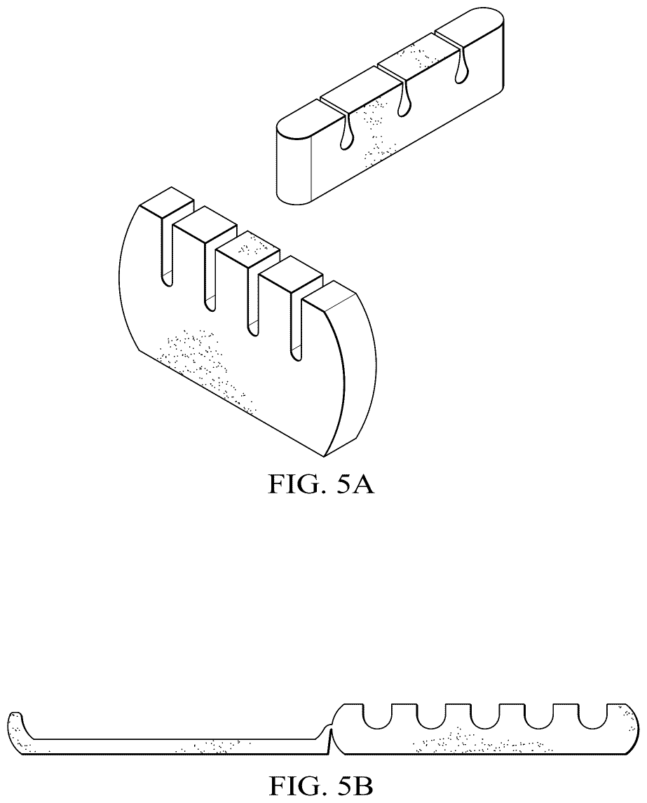

FIGS. 5A and 5B show a clamp, according to an embodiment of the disclosure. Unlike prior configurations, the clamp may be made of material that softly interacts with lines. As an example, at least the grooves holding the lines in place can be made of a neoprene or flexible foam material. FIG. 5A show two different configurations.

FIG. 5B shows a configuration with a lid connected a hinge that sits on top of the lines to ensure they maintain their place.

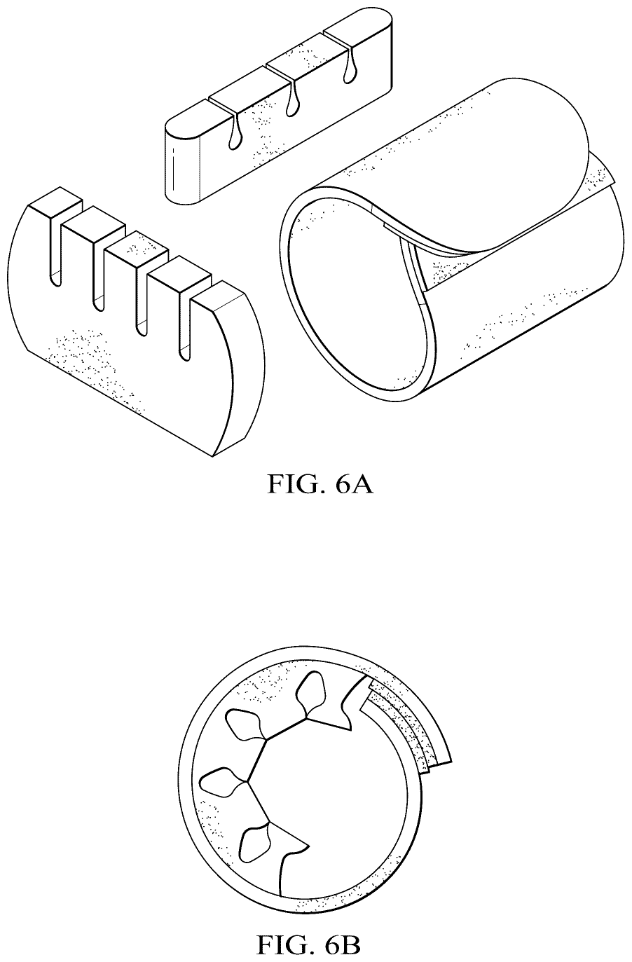

FIGS. 6A and 6B show a clamped sleeve design, according to an embodiment of the disclosure. With reference to 6A, the clamp design can be integrated into the rollable sleeve. With reference to FIG. 6B, one can see how the rolling of the clamp secures a line in place. The design of FIGS. 6A and 6B can be combined with any of the feature described above with reference to other figures. For example, as referenced above, the surfaces of the wrapping material may have different configurations. The flexible clamp design is but one example.

FIGS. 7A through 7D show a configuration of using individual sleeves for lines to attract multiple lines together.

FIG. 7A shows a sleeve 730 for a line. The sleeve includes a layer 735, a magnet 732, and connecting portions 737, 739. In particular configurations, the layer 735 may be flexible enough to wrap around the line but prevent pinching of the line. In other configurations, the layer 735 may be a rigid design that has a curved shell to fit around the line and definitively prevent a pinching through the specific structure of the layer. Any suitable plastic may be used for such a rigid design.

The magnet 732 is generally attracted to a corresponding a magnet or electromagnet described with reference to FIG. 7B.

The connection portions 737, 739 generally connect to another to secure the sleeve 730 on the line. Magnets, hook and loop fasteners, and knobs may be used-among others.

FIG. 7B shows a sleeve 750 used to collect multiple sleeves 730. The sleeve includes a layer 755, attracting layer 752, and connecting portions 757, 759. In particular configurations, the layer 755 may be flexible enough to wrap around the line but prevent pinching of the line. In other configurations, the layer 755 may be a rigid design that has a curved shell to fit around the line and definitively prevent a pinching through the specific structure of the layer. Any suitable plastic may be used for such a rigid design.

The attracting layer 752 magnetically attracts the magnets 732 of FIG. 7A of multiple different sleeves 730. Such magnetic attractions assist in gathering multiple lines for securing in the sleeve 730. In some configurations, the magnetic force may be large enough such that the sleeve 730 “snaps” into place. In some configurations, the attracting layer 752 is a permanent magnet. In other configurations, the attracting layer 752 is an electromagnet that is selectively powered by a suitable power source such as battery or the like (not shown). Selective magnetic attraction can be used in some configurations to collect and group multiple lines in a sleeve 750 during attraction (power is one) and then remove lines from a sleeve when there is a lack of attraction (power is off).

The connection portions 737, 739 generally connect to one another to secure the sleeve 730 on the line. Magnets, hook and loop fasteners, and knobs may be used-among others.

FIG. 7C shows respective lines 700A, 700B, and 700C having sleeves 730A, 730B, and 730C on them. FIG. 7D shows the sleeves 730A, 730B, and 730C of lines 700A, 700B, and 700C being secured in 750. FIG. 7E shows a configuration in which the sleeves 730A, 730B, and 730C magnetically attract to one another. In such a configuration, the sleeve 750 with the attracting layer 752 may not be used. Rather, with the configuration of FIG. 7E, either another sleeve may be used or no sleeve at all. In configurations where no sleeve is used, the magnetic attraction in the sleeves 730A, 730B, and 730C may keep the line coupled to one another.

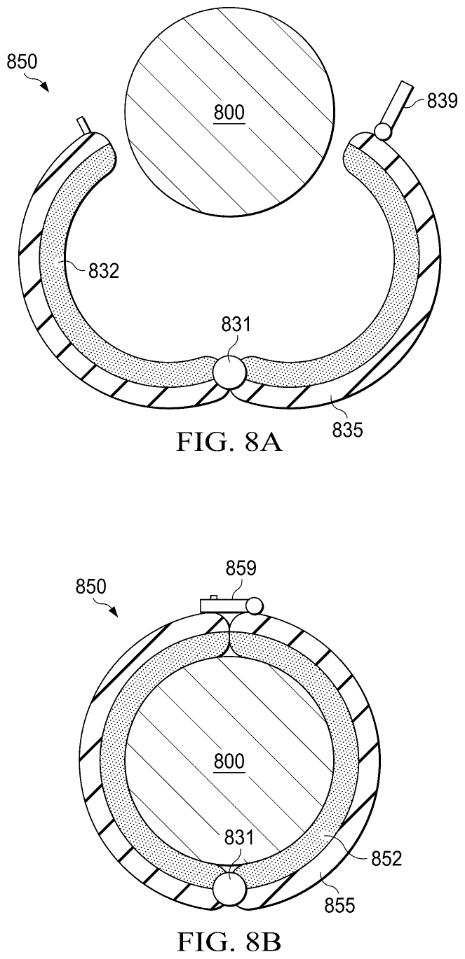

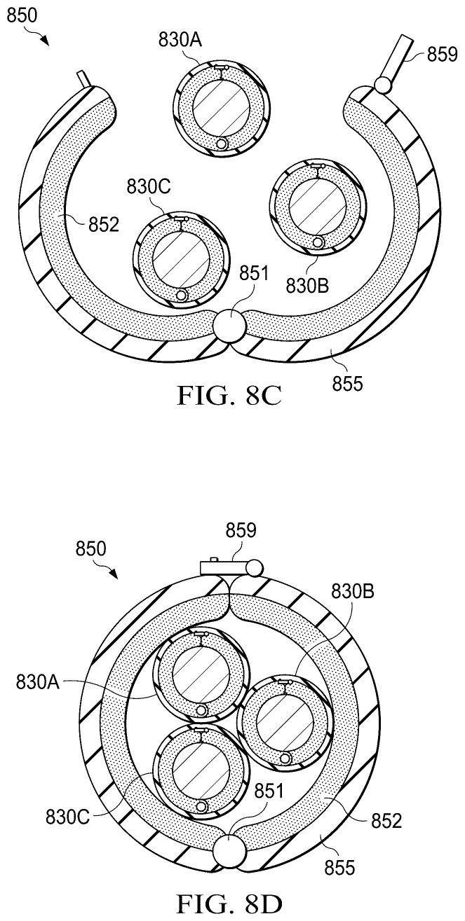

FIGS. 8A through 8D show rigid structure sleeves that may be used in particular configurations. Such rigid structures may be used to protect the lines from being pinched—even from other lines grouped together. While a round shape for such structures are shown in the figures, other shapes may be utilized.

FIGS. 8A and 8B show a sleeve 830 with a shell 835, inner liner 832, and connection mechanisms 839, 831. The sleeve 830 surrounds the line 800. FIG. 8A shows placement of the line 800 in the sleeve whereas FIG. 8B shows the sleeve connected to the line 800.

The shell 835 may be made of any suitable material that is configured to resist a pinching of the line 800 when a force is imparted thereon. Non-limiting examples include plastics and metals. As referenced above, in particular configurations, a sleeve may have material that magnetically attracts to other materials. In some configurations, the material uses in assisting in the rigidity of the shell may serve a dual purpose in all being magnetically attracted to another material.

The inner liner 832 is configured to interact with line 800 and generally prevent slipping of the sleeve up and down the line. In particular configurations, the inner liner 832 is made of a foam or gelled material that changes shape, but does not adversely pinch the line 800.

The connection mechanisms 839, 831 are a bottom hinge and a hinged clasp. However, any other suitable connection mechanism may be utilized-including magnets and hook and loop fasteners.

FIGS. 8C and 8D show a sleeve 850 housing multiple sleeves 830A, 830B, and 830C, which may be formed as shown in FIGS. 8A and 8B. The sleeve 850 includes a shell 855, inner liner 852, and connection mechanisms 859, 851. FIG. 8C shows placement of sleeves 830A, 830B, and 830C in sleeve 850 whereas FIG. 8D shows the sleeve connected to the line 800.

The shell 855 may be made of any suitable material. Like shell 835 of FIG. 8A, shell 855 may be rigid; however, when shell 835 is already used, shell 855 has less concern on a prevention of pinching of the lines. Non-limiting examples include plastic and rigid fabrics.

The inner liner 852 is configured to interact with sleeves 830A, 830B, and 830C. The material of the inner liner 852 in particular configurations may be any material to facilitate the magnetic interactions. Yet other material may be used-including those similar to inner liner 852.

The connection mechanisms 859, 851 are a bottom hinge and a hinged clasp. However, any other suitable connection mechanism may be utilized-including magnets and hook and loop fasteners.

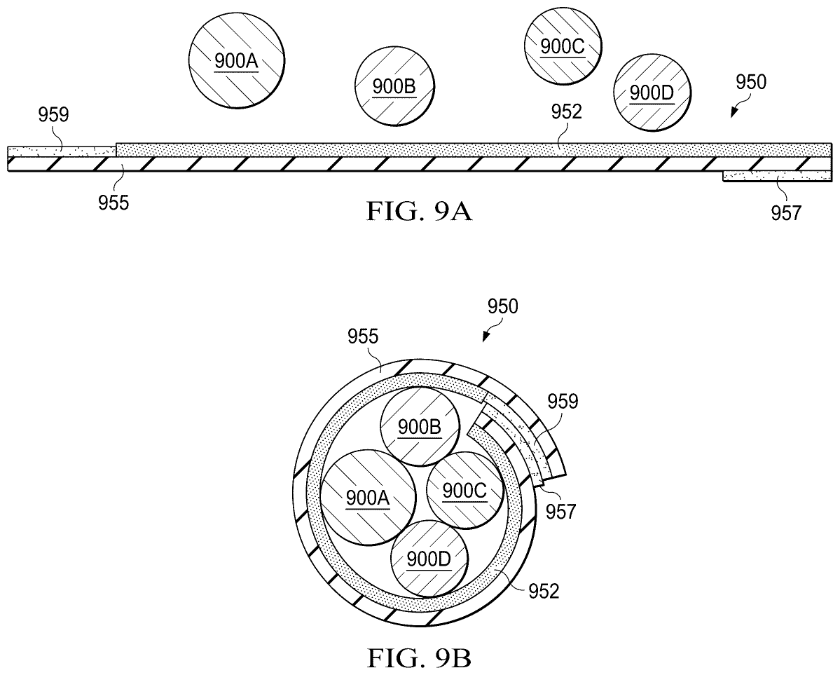

FIGS. 9A and 9B illustrate the placement of a sleeve 950 around a plurality of lines 900A, 900B, 900C, and 900D. The sleeve a first layer 955, a second layer 952, and connecting portions 957, 959. While two layers are shown here, three or more layers may be used in some configurations; and, other configurations may have only a single layer.

In the configuration of FIG. 9A, the first layer 955 generally creates the structure of the sleeve 950. The second layer 952 is designed to interact with the lines 900A, 900B, 900C, and 900D. The material of the second layer 952 can be chosen to prevent slippage of the sleeve with respect to the lines and/or prevent pinching of the lines 900A, 900B, 900C, and 900D. The surface of the second layer may include bumps or channels to assist with the interaction of the lines. Additionally, the surface may include material that grips the corresponding material of the lines. Non-limiting examples of material for the second layer include, but are not limited, to foam, gel, soft fabrics, and soft plastics.

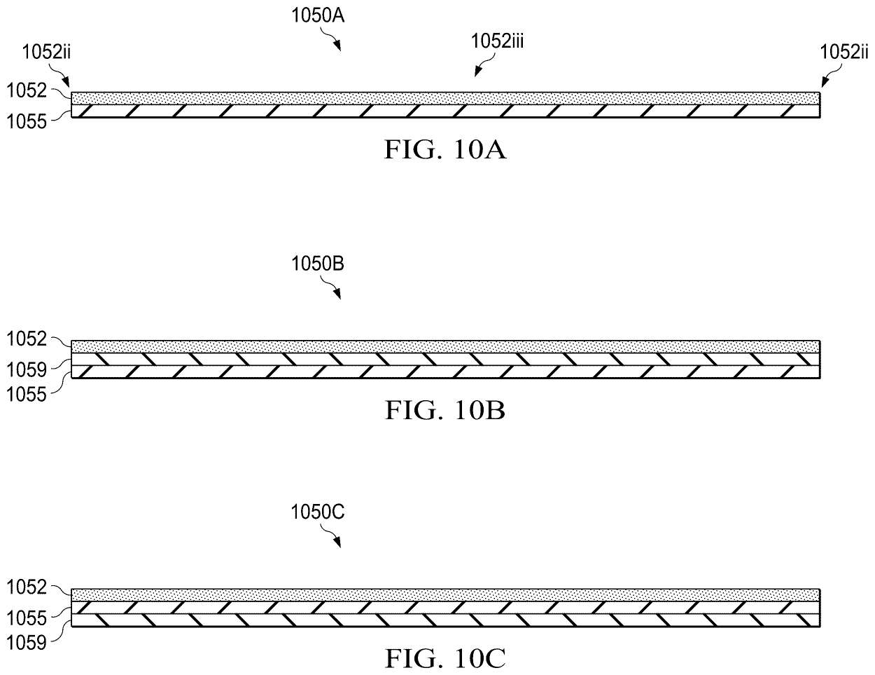

FIGS. 10A, 10B, and 10C illustrate a modular nature of layers, according to an embodiment of the disclosure. With reference to FIG. 10A, a sleeve 1050A has a first layer 1055 and a second layer 1052 that are removable from one another, for example, using hook and loop fasteners. Such removability assists in “changing out” the second layer for a different material to interact with the lines. Alternatively, the first layer 1055 may be changed out the connection mechanisms used for the sleeves. In yet additional configurations, an additional layer may be added with new features for the sleeve.

In embodiments of the disclosure, a layer such as 1055 of FIG. 10A has corresponding hook and look fastener configurations on opposite surfaces (e.g., top and bottom). Accordingly, the design can easily wrap around an object and connect to itself. One or multiple subsequent layers can be added to either side of the first layer. Each subsequent layer can have a corresponding hook and loop design to at least connect to the first layer. In some configurations, some of the additional layers may also have their own hook and loop configurations on both respective surfaces to allow further layers. In other configurations (e.g., a finishing surface designed for no additional surfaces), the corresponding hook and loop fasteners may not be contained on the entire surface, but either sized as to not interrupt the connection mechanism or replace it. As an example of the preceding sentence, the entire top surface of the first layer 1055 may have a hook and loop design. The second layer 1052 layer may be added to the top as a soft flexible surface to interact with lines. To retain the hook and loop functionality, the new layer must leave the existing hook and loop fastener design exposed or have its own hook and loop fastener. As a replacement design, the second layer 1052 need not have the corresponding hook and loop fasteners across its entire surface; rather, more corresponding hook and loop fastener material may be included at the edges (e.g., 1052i and 1052ii) to receive the corresponding surface from the first layer 1055 after wrapping. The center portion 1052iii may include some hook and loop fastener material—enough for another layer.

With reference to FIG. 10B, the first layer 1055 and the second layer 1052 may be disconnected from one another to allow placement of a third layer 1059 therebetween. With such a configuration, the sleeve 1050A can be upgraded to the sleeve 1050B.

With reference to FIG. 10C, the third layer 1059 is connected under the first layer 1055 as opposed to between the first layer and 1055 and second layer 1052. With such a configuration, the sleeve 1050A can be upgraded to the sleeve 1050B. As above, the third layer 1059 may include appropriate hook and fastener materials to preserve the connection of the sleeve 1050C. With such a configuration, the sleeve 1050A can be upgraded to the sleeve 1050C.

Although this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure,

What is claimed is:

1. A wrap for collecting a plurality of lines, cords, tubes, or wires, the wrap comprising:
a first layer having a first connecting mechanism on a first surface of the first layer and a second connecting mechanism on a second surface of the first layer, wherein
the first surface and the second surface are on opposite surfaces of the first layer such that one may be viewed as being on a top of the first layer and the other viewed as being on a bottom of the first layer when the wrap is in an unwrapped state,
the first connection mechanism is removably attachable to the second connection mechanism to transition the wrap between the unwrapped state and a wrapped state,
the first layer is configured to wrap around the plurality of lines, cords, tubes, or wires when the wrap is in a wrapped state, and
a portion of the first layer overlaps another portion of the first layer when the wrap is in a wrapped state.

2. The wrap of claim 1, wherein the first connecting mechanism and the second mechanism are configured to allow variance in the amount of the portion of the first layer overlapping another portion of the first layer when the wrap is in the wrapped state.

3. The wrap of claim 1, wherein the plurality of lines, cords, tubes, or wires are allowed to contact one another when the wrap is in the wrapped state.

4. The wrap of claim 1, wherein the first connecting mechanism and the second mechanism connect with hook and loop fasteners.

5. The wrap of claim 1, wherein the first connecting mechanism and the second mechanism connect with magnets.

6. The wrap of claim 1, further comprising:
a second layer configured to interact with the plurality of lines, cords, tubes, or wires when the apparatus is in the wrapped state.

7. The wrap of claim 6, wherein the second layer is attachably removable from the first layer.

8. The wrap of claim 6, wherein the second layer is configured to prevent slippage of the wrap with respect to the plurality of lines, cords, tubes, or wires when the wrap is in the wrapped state.

9. The wrap of claim 1, wherein the first layer is reinforced with wires configured to resist unwrapping once the wrap is in the wrapped state.

10. The wrap of claim 1, wherein the first layer contains a flexible material.

11. The wrap of claim 1, wherein the first layer contains at least one of fabric or neoprene material.

12. The wrap of claim 1, wherein the first layer is configured to interchangeably wrap around the plurality of lines, cords, tubes, or wires such that either the top or the bottom of the first layer is facing outwards when the wrap is in a wrapped state.

13. The wrap of claim 1, wherein at least a portion of the first layer includes material configured to protect the plurality of lines, cords, tubes, or wires from pinching when force is imparted on the wrap in the wrapped state.

14. A wrap for collecting a plurality of tubes, the wrap comprising:
a first layer having a first connecting mechanism on a first surface of the first layer and a second connecting mechanism on a second surface of the first layer, wherein
the first surface and the second surface are on opposite surfaces of the first layer such that one may be viewed as being on a top of the first layer and the other viewed as being on a bottom of the first layer when the wrap is in an unwrapped state,
the first connection mechanism is removably attachable to the second connection mechanism to transition the wrap between the unwrapped state and a wrapped state,
the first layer is configured to wrap around a plurality of lines, cords, tubes, or wires when the wrap is in a wrapped state,
a portion of the first layer overlaps another portion of the first layer when the wrap is in a wrapped state,
the first connecting mechanism and the second mechanism are configured to allow variance in the amount of the portion of the first layer overlapping another portion of the first layer when the wrap is in the wrapped state, and
the plurality of lines, cords, tubes, or wires are allowed to contact one another when the wrap is in the wrapped state.

15. The wrap of claim 14, wherein the first layer is configured to interchangeably wrap around the plurality of lines, cords, tubes, or wires such that either the top or the bottom of the first layer is facing outwards when the wrap is in a wrapped state.

16. The wrap of claim 14, wherein the first connecting mechanism and the second mechanism connect with hook and loop fasteners.

17. The wrap of claim 14, wherein the first connecting mechanism and the second mechanism connect with magnets.

18. The wrap of claim 14, further comprising:
a second layer configured to interact with the plurality of lines, cords, tubes, or wires when the apparatus is in the wrapped state.

19. The wrap of claim 18, wherein the second layer is attachably removable from the first layer.

20. The wrap of claim 14, wherein the first layer contains a flexible material.
